(19) United States
(12) Patent Application Publication
(10) Pub. No.: US 2006/0148021 A1
(43) Pub. Date: Jul. 6, 2006

(54) Extraction of a proteinaceous macromolecule from a biological sample

(75) Inventor: Benjamin Ross Herbert, North Epping (AU)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Proteome System Intellectual Property Pty Ltd

(21) Appl. No.: 10/533,742

(22) PCT Filed: Nov. 3, 2003
(86) PCT No.: PCT/AU03/01448

§ 371 Date: Mar. 3, 2006

(30) Foreign Application Priority Data
Nov. 4, 2002 (AU) 2002952533

(51) Int. Cl. C12Q 1/37; C07K 14/47

(52) U.S. Cl. 435/23; 530/412

(57) ABSTRACT

The invention provides a method of treating a biological sample to extract a macromolecule under acidic conditions. In one aspect the present invention provides a method for enhancing the solubilisation of at least one proteinaceous macromolecule in a biological sample without inducing substantial acid hydrolysis of said proteinaceous macromolecule, said method comprising incubating the biological sample in a solubilisation reagent at a pH between about pH 1.0 and about pH 6.0. The invention also provides novel reagents and kits for extraction of macromolecules from biological samples.

### FIELD OF THE INVENTION

The present invention is in the field of extraction of proteinaceous macromolecules, more particularly with regard to the improved solubilisation, recovery, and analysis of proteins from biological samples.

### BACKGROUND OF THE INVENTION

Solubilisation of proteins from biological samples is a difficult and tedious process and to date has only been partially successful. Problems are particularly evident with some micro-organism samples which show poor resolution and streaking following extraction and resolution by two-dimensional gel electrophoresis.

Most proteins have acidic isoelectric points and thus are anions (and repel each other in solution) at alkaline pH. Conventional wisdom suggests that alkaline pH favours protein solubility. Accordingly, conventional protein extractions are done at alkaline pH (between pH 8 and 10), using Tris (alkaline) buffer, to ensure good protein solubility. For example, soluble protein samples can be readily taken up in the most commonly used solubilisation sample solution of 8M urea, 4% 3-[(3 cholamidopropyl)dimethylammonio]-1-propane sulphonate (CHAPS), 50-100 mM dithiothreitol (DTT) and 40 mM Tris. With regard to gel electrophoresis, sample preparation may also require an alkaline pH to ensure efficient disulfide bond reduction with reagents such as dithiothreitol. Failure to reduce and alkylate proteins prior to isoelectric focussing can result in substantial loss of resolved proteins.

The standard solubilisation solution is not, however, ideal for many proteins and the challenge is to improve the solubilisation and separation of insoluble samples such as membrane and membrane-associated proteins and proteins from tissues like hair and skin.

New methods for enhancing protein solubility which include introducing new reagents such as thiourea, (to denature and unfold a protein) sulfobetaine surfactants and tributyl phosphine into the IEF sample solution, have been described in, for example,
Ravilloud, et al, Electrophoresis 1997, 18, 307-316;
Chevallet et al, Electrophoresis 1998, 1901-1909; and
Herbert B R., Electophoresis 1998, 19, 845-851.

These methods, however do not address some of the most common causes of poor extraction which come from the biological sample itself, such as nucleic acids, salts, lipids, pigments and in the case of microorganisms, the cell wall, which are also solubilised by the standard solutions described above. Cell walls of fungi share with plant and bacterial cell walls, and also with extracellular matrix material of mammalian cells, an anionic surface and a reliance on 1,4- and 1,3-linked polysaccharides as fibrous components. These glycans have all the non-hydrogen ring constituents in an equatorial position and form ribbon-like (cellulose and chiton) or helical structures. Solubilised cell wall polysaccharides behave as polyanions and are able to bind many proteins via electrostatic interactions, and as such they can pose a major problem in the extraction of proteins from microorganisms.

In isoelectric focussing, large polysaccharides can block the pores of the gels and make protein entry difficult. Furthermore, during isoelectric focusing proteins bind to polysaccharides and focus poorly with considerable horizontal streaking often observed at the acidic end of a two-dimensional gel.

General Information.

As used herein the terms “derived from” or “derivative” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment and, in particular, with respect to an apparatus or a method of assaying shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally, equivalent products, compositions and methods are clearly within the scope of the invention, as described herein.

The present invention is performed without undue experimentation using, unless otherwise indicated, conventional techniques of molecular biology, proteomics, electrophoresis, and gel technology. Such procedures are described, for example, in the following texts that are incorporated by reference:
Sambrook, Fritsch & Maniatis, Molecular Cloning: A Laboratory Manual, Cold Spring Harbor Laboratories, New York, Second Edition (1989), whole of Vols I, II, and III.

### SUMMARY OF THE INVENTION

In developing the present invention, the inventors have sought to obtain an improved method for the extraction of a proteinaceous macromolecule from a biological sample. To this end, the inventors have developed a method of treating a biological sample to solubilise a proteinaceous macromolecule under acidic conditions, wherein acid hydrolysis of the proteinaceous macromolecule does not substantially occur.

As used herein the term “proteinaceous macromolecule” is preferably a recombinant or non-recombinant protein, glycoprotein, polypeptide, peptide or derivative or fragment thereof.

As used herein the term “acidic conditions” refers to a pH of less than about pH 7, more preferably less than about pH 6. Acidic conditions can be provided by any acid reagent under conditions such that acid hydrolysis of the proteinaceous macromolecule does not substantially occur. Preferably, the acidic conditions prevent cell wall solubilisation.

As used herein, the term “acid hydrolysis” refers to the effect of an acid on a protein to cause the hydrolysis of peptide bonds and thus the breakdown of the protein into its amino acid constituents. Acid hydrolysis of a protein will usually occur if a protein is treated with a strong acid particularly at temperatures above room temperature. Strong acid hydrolysis (e.g., 6N HCl; @ 110° C.) also causes the amide amino acids Asparagine and Glutamine to be converted to their acids, Aspartate and Glutamate respectively, and can destroy Tryptophan.

According to the present invention, acid hydrolysis of a macromolecule in the biological sample does not substantially occur. Preferably, the method of the invention provides several more advantages over existing methods of extraction of proteins from biological samples. For example, preferably the method minimises removal of a sugar or sugars from the backbone of the protein during the solubilisation process. Further, preferably cell debris is more easily removed during the purification process, as the acidic conditions preferably prevent solubilisation of a cell membrane or cell wall. Preferably, there is no requirement to precipitate the protein prior to solubilisation. Preferably the solubility of alkaline proteins is enhanced.

Accordingly, in a first aspect the present invention provides a method for enhancing the solubilisation of at least one proteinaceous macromolecule in a biological sample without inducing substantial acid hydrolysis of said proteinaceous macromolecule, said method comprising incubating the biological sample in a solubilising reagent at a pH between about pH 1.0 and about pH 6.0.

In a preferred embodiment, the solubilising reagent has a pH of between about pH 1.0 and about pH 6.0.

In one embodiment of the invention a proteinaceous macromolecule is solubilized using conditions that comprise incubating the proteinaceous macromolecule at a pH below the isoelectric point of the proteinaceous macromolecule and preferably at least about 1 pH unit or about 2 pH units or about 3 pH units or about 4 pH units or about 5 pH units or about 6 pH units or about 7 pH units or about 8 pH units below the isoelectric point of the proteinaceous macromolecule. Preferably, the method further comprises the first step of determining the isoelectric point of the proteinaceous macromolecule.

Preferably, the proteinaceous macromolecule is a polypeptide or complex or aggregate thereof, such as, for example, a homo-multimeric or hetero-multimeric enzyme comprising the polypeptide, said polypeptide or complex or aggregate thereof having an alkaline isoelectric point i.e., an isoelectric point (pI) above about 7.0.

In a preferred embodiment the biological sample is subjected to a physical or chemical means to disrupt the biological sample, thereby producing a proteinaceous extract.

In one embodiment, the biological sample is subjected to a physical or chemical means to disrupt the biological sample in the presence of the solubilising reagent at a pH between about pH 1.0 and about pH 6.0.

In an alternate embodiment, such as, for example, in the case of a protein that is localized internal to a cell, organelle or prolamellar body or intrinsic to a cell membrane, it is preferred to subject the biological sample to the physical or chemical means to disrupt the biological sample before the biologic sample is incubated with the solubilising reagent at a pH between about pH 1.0 and about pH 6.0. In this way, exposure of the protein of interest to the solubilising reagent is optimized, thereby enhancing solubilization and, if appropriate, subsequent recovery, of the protein.

Preferably, the method further comprises recovering the at least one proteinaceous macromolecule. The proteinaceous macromolecule may be recovered in soluble form, such as, for example, in the presence of a solubilising reagent having a pH between about pH 1.0 and about pH 6.0, or alternatively, following further treatment by one or more means known to a skilled protein chemist e.g., precipitation, concentrations ultrafiltration, membrane filtration or fractionation.

In one embodiment, the solubilized proteinaceous macromolecule is recovered by performing a process comprising precipitating the at least one solubilised macromolecule such as, for example, using ammonium sulfate or polyethylene glycol (PEG) or an organic solvent such as acetone or methanol to thereby form a protein precipitate.

In another preferred embodiment recovering comprises precipitating and resuspending the protein precipitate.

Alternatively, or in addition, the inventive method further comprises reducing and alkylating the solubilized proteinaceous macromolecule.

In another preferred embodiment, the solubilized proteinaceous macromolecule is subjected to a resolving means for a time and under conditions sufficient to resolve the solubilised macromolecule from other macromolecules present in the biological sample.

In yet another embodiment, the solubilized proteinaceous macromolecule is subjected to a resolving means for a time and under conditions sufficient to resolve the solubilised macromolecule from other macromolecules present in the biological sample wherein the resolved protein is identified by a means of protein or proteome analysis such as, for example, an analytic method known to a skilled protein chemist, to thereby determine one or more physicochemical properties of the resolved protein (eg., isoelectric point, molecular weight, amino acid composition, amino acid sequence) or one or more enzymatic properties of the resolved protein (eg., by bioinformatics analysis of the amino acid sequence of the protein and comparison to the properties of one or more known proteins). Preferably resolving and analysing the at least one macromolecule comprises using proteomic techniques.

In one particularly preferred embodiment, the present invention provides a method of solubilising at least one proteinaceous macromolecule in a biological sample without inducing substantial acid hydrolysis of said proteinaceous macromolecule, said method comprising:
(i) subjecting the biological sample to a physical or chemical means to disrupt said biological sample an incubating the biological sample in the presence of a reagent at a pH between about pH 11.0 and about pH 6.0 to thereby soluble at least one proteinaceous macromolecule in the biological sample; and
(ii) performing one or more processes selected from the group consisting of:
   (a) recovering the solubilized proteinaceous macromolecule by performing a process comprising precipitating one or more proteins in the extract at (i) to thereby precipitate at least the solubilised proteinaceous macromolecule and resuspending the precipitated proteinaceous macromolecule;
   (b) reducing and alkylating the solubilized proteinaceous macromolecule at (i) or the resuspended proteinaceous macromolecule at (ii)(a); and
   (c) subjecting the solubilized proteinaceous macromolecule at (i) or the resuspended proteinaceous macromolecule at (ii)(a) or the reduced and alkylated proteinaceous macromolecule at (ii)(b) to a resolving means for a time and under conditions sufficient to resolve the proteinaceous macromolecule from other macromolecules present in the biological sample and then identifying the resolved proteinaceous macromolecule.

In an alternative preferred embodiment the present invention provides a method of solubilising at least one proteinaceous macromolecule in a biological sample without inducing substantial acid hydrolysis of said proteinaceous macromolecule, said method comprising:
(i) subjecting the biological sample to a physical or chemical means to disrupt said biological sample, thereby producing a proteinaceous extract;
(ii) incubating the proteinaceous extract in the presence of a reagent having a pH between about pH 1.0 and about pH 6.0 to thereby solubilize at least one proteinaceous macromolecule in the extract; and
(iii) performing one or more processes selected from the group consisting of:
   (a) recovering the solubilized proteinaceous macromolecule by performing a process comprising precipitating one or more proteins in the extract at (ii) to thereby precipitate at least the solubilised proteinaceous macromolecule and resuspending the precipitated proteinaceous macromolecule;
   (b) reducing and alkylating the solubilized proteinaceous macromolecule at (ii) or the resuspended proteinaceous macromolecule at (iii)(a); and
   (c) subjecting the solubilized proteinaceous macromolecule at (ii) or the resuspended proteinaceous macromolecule at (iii)(a) or the reduced and alkylated proteinaceous macromolecule at (iii)(b) to a resolving means for a time and under conditions sufficient to resolve the proteinaceous macromolecule from other macromolecules present in the biological sample and then identifying the resolved proteinaceous macromolecule.

The methods of the present invention are particularly suitable for the recovery of proteins from cell membranes or high molecular weight proteins that are relatively recalcitrant to known solubilization means without incurring significant protein degradation, shearing or degradation. Accordingly in a preferred embodiment, the present invention provides a method for the solubilization of a protein selected from the group consisting of a mucinous protein, a cell wall protein (i.e. intrinsic to the cell wall), a membrane protein (i.e. intrinsic to the cell membrane), a cell wall-associated protein (i.e. extrinsic to the cell wall) and a membrane-associated protein (i.e. extrinsic to the cell membrane).

In a second aspect, the invention provides a kit for enhancing solubilisation of a proteinaceous macromolecule a biological sample without inducing substantial acid hydrolysis of said proteinaceous macromolecule, the kit comprising a solubilisation reagent to solubilise at least one macromolecule in a biological sample, wherein the solubilisation reagent has a pH of about pH 1 to about pH 6 and optionally comprising directions to solubilise and/or recover a macromolecule in a biological sample, and/or directions to resolve a macromolecule in a biological sample.

In a third aspect, the invention provides a macromolecule obtained by the method of the first aspect.

The reagent may be known per se but for the use according to the present invention at the pH range specified above. In another aspect, the invention provides use of a reagent having a pH of about pH 1 to about pH 6 in the preparation of an solubilisation reagent solution for use in solubilising a proteinaceous macromolecule from a biological sample.

### BRIEF DESCRIPTION OF THE FIGURES

FIG. 1 is a photographic copy of a comparison of 2D gels of E. coli proteins separated by alkaline (a) or acidic (b) extractions. Note significant smearing in the acidic half of the alkaline extract gel, and its absence in the gel from the acidic extract.

FIG. 2 is a photographic copy of a comparison of 2D gels of B. subtilis proteins separated by alkaline (a) or acidic (b) extractions. Note the almost complete lack of resolved protein spots in the alkaline extract gel. The acidic extract gel however displays good protein resolution with an almost complete lack of streaking.

FIG. 3 is a photographic copy of a comparison of 2 D gels of S. cerevisiae separated by alkaline (a) or acidic (b) extractions. Note the detergent smear present in the alkaline extract is absent in the acidic extract, the increased yield of alkaline proteins in the acidic extract, and the reduced horizontal streaking at the far acidic end of the acidic extract gel.

FIG. 4 is a photographic copy of a comparison of 2D gels of T. harzianum separated by alkaline (a) or acidic (b) extractions. Note the reduced streaking in the acidic extract gel.

FIG. 5 is a photographic copy of a 2D gel of T. harzianum separated by acidic extraction, with the addition of protease inhibitors. Note the increased yield of high molecular weight proteins compared to FIG. 4.

FIG. 6 is a photographic copy of a comparison of the effect of using different acidic extraction solutions on the 2D electrophoretic separation of S. cerevisiae proteins. Note the change in protein yield observed when ascorbic acid (a) and orthophosphoric acid pH 3 (b) are used.

FIG. 7 is a photographic copy of an aloian blue stained electroblot of 1D SDS-AgPAGE of B. subtilis extracts. Lanes 1 and 6 contain Mw markers of 220 kDa, indicated by arrows. Lane 7 has a positive mucin control (Muc2), with the arrow pointing to ˜4 MDa. Lanes 2,3 are <100 kDa fractions, and display no staining. Lanes 4, 5 are the >100 kDa fraction, showing stained smears, indicating the presence of large, acidic molecules. Three bands of unknown composition are indicated by arrows.

FIG. 8 is a photographic copy of a comparison of 2D gels of >100 kDa (a) and <100 kDa (b) fractions of a B. subtilis extract. Note the absence of streaking in the <100 kDa fraction gel, and the similarity in poor resolution of the >100 kDa extract with the unfractioned sample (FIG. 2a).

### DETAILED DESCRIPTION OF THE INVENTION

In a first aspect the present invention provides a method for enhancing the solubilisation of at least one proteinaceous macromolecule in a biological sample without inducing substantial acid hydrolysis of said proteinaceous macromolecule, said method comprising incubating the biological sample in a solubilisation reagent at a pH between about pH 1.0 and about pH 6.0.

In a preferred embodiment, the solubilisation reagent has a pH of between about pH 1.0 and about pH 6.0.

Biological Samples and Proteinaceous Macromolecules

As used herein the term “biological sample” refers to any sample comprising a recombinant or non-recombinant proteinaceous macromolecule. Preferably, the biological sample comprises cellular or tissue material or a culture or fraction thereof. For example, preferably the biological sample is a microorganism sample (eg., bacterial, yeast or fungal), an insect cell sample (eg., exoskeleton sample), a plant cell sample or seed, an animal cell sample or tissue sample (eg. skin and hair samples), or fractions thereof.

In a preferred embodiment the protein is an alkaline protein. By “alkaline protein” is meant a protein having an isoelectric point above about 7.0. The isoelectric point of a protein may be predicted from the amino acid sequence of the protein or alternatively it may be determined empirically by isoelectric focussing or reverse phase chromatography. Preferred alkaline proteins are selected from the group consisting of alpha and beta chains of haemoglobin, immunoglobulins (eg., antibodies) and ribosomal proteins, cell wall and cell wall associated proteins, membrane and membrane associated proteins, and proteins derived from tissues eg. skin and hair.

In a particularly preferred embodiment the alkaline protein is a cell wall or cell membrane protein or protein associated with the cell wall or membrane fraction of a cell lysate that is relatively insoluble in aqueous solution containing 7M urea, 2M thiourea and 1% (w/w) C7 (pH 8.0-8.5) or equivalent thereof.

Disruption

In a preferred embodiment, the biological sample is subjected to a physical or chemical means to disrupt the biological sample. Preferably, the method is performed such that artefactual proteolysis and acid hydrolysis is minimised and substantially does not occur.

As used herein, the term “disruption” refers to breaking apart or breakdown of a biological sample. For example disruption of the biological sample refers cell lysis, breakdown of tissue or breakdown of cell walls or membranes.

Methods of disruption vary according to the nature of the sample, and typically include autolysis (eg. by incubation with toluene or a buffer), enzymatic lysis (eg. with lysozyme or β-glucanase), grinding, liquid nitrogen cooling glass-bead beating, or sonication.

In a preferred embodiment of the present invention, the sample is disrupted by sonication. In a more preferred embodiment the sample is disrupted by sonication at least partly in the presence of the solubilisation reagent at a pH of between about pH 1.0 and about pH 6.0.

It is understood that the biological sample can be disrupted in the presence or absence of the solubilising reagent. In one embodiment the biological sample is disrupted in the presence of a buffer. Further it is also within the scope of the invention that the biological sample is disrupted at least partly in the presence of the solubilising reagent at a pH of between about pH 1.0 and pH 6.0.

As the risk of artefactual proteolysis (i.e., proteolysis by the proteins own enzymes) is increased following cell disruption, it is preferable that the time between disruption and treating the sample with the reagent is minimised.

Solubilisation Reagent

As used herein the term “incubating” includes contacting, treating, suspending, in the presence of, or the like, for such a time and under conditions in this invention for solubilisation to occur.

In one embodiment the solubilisation reagent comprises a detergent. Preferably, the detergent enhances disruption of a cell membrane or cell wall, and more preferably, the detergent solubilises or partially solubilises a proteinaceous macromolecule in the biological sample. Preferably, the detergent is selected from the group consisting of an ionic detergent cationic detergent, anionic detergent, or non-ionic detergent. Examples of well known detergents useful according to the present invention include 3-(4-heptyl) phenyl 3-hydroxypropyl dimethyl amminio propane sulfonate (C7Bz0), and tetradecanoylamido propyl dimethyl ammonio propane sulfonate (ASB 14).

In another embodiment of the invention, the solubilising reagent comprises a chaotropic agent, which preferably helps to denature, unfold and solubilise a proteinaceous macromolecule in the biological sample. Preferably the chaotropic agent is selected from the group consisting of urea, or thiourea, or a mixture thereof. The concentration of the chaotropic agent can be adjusted as desired. It is understood, however that the use of a chaotropic agent my not be useful in the food industry and alternate agents to effect solubilisation may be included in the solubilisation agent.

In a preferred embodiment, the biological sample is treated with a solubilisation reagent having a pH of between about pH 1 and about pH 6, and which comprises a detergent and/or a chaotropic agent. According to one embodiment, a solubilising reagent is prepared and the pH of the solubilising reagent is adjusted to have a pH of between about pH 1 and about pH 6.

Alternatively, the pH of the sample is adjusted to have a pH of between 1 and about pH 6.

Preparation of biological sample may also comprise inactivation or removal of interfering substances, such as for example, proteolytic enzymes. Proteolytic enzymes can be inactivated, for example, by the addition of urea, thiourea, DTT, proteinase inhibitors, Tris base, acetone precipitation, or by boiling the sample in SDS buffer.

In one embodiment, a protease inhibitor is added to the reagent or biological sample.

Preferably, the biological sample is incubated in a solubilising reagent at a pH of between about pH 1.0 and about pH 6.0 for a time and temperature suitable for solubilisation of a proteinaceous macromolecule while minimising the effects of acid hydrolysis on the proteinaceous macromolecule.

Acid Reagent and Conditions

In one embodiment, the biological sample is incubated in a solubilisation reagent at a pH of less than about pH 7. More preferably, the biological sample is incubated in a solubilisation reagent at a pH of less than about pH 6 more preferably less than about pH 5.0, more preferably between about pH 2.0 and about pH 5.0, more preferably between about pH 3.0 and pH 4.0. In a preferred embodiment the solubilisation reagent has a pH of less than about pH 6.0, more preferably less than about pH 5.0. In one embodiment the solubilisation reagent has a pH of about pH 2 to about pH 5. In another embodiment, the solubilisation reagent has a pH of about pH 3 to about pH 4. In another embodiment the solubilisation reagent has a pH of about pH 2 to about pH 3.

The pH of a solution can be determined by any means known to a person skilled the art. It will be recognised by a person skilled in the art that pH measurements can vary and the term “about” as used in this regard reflects that variation.

In one embodiment, the pH of the solubilisation reagent or biological sample is adjusted by the presence of an acidic reagent. In one embodiment the acidic reagent comprises an aqueous solvent such as an acidic aqueous solvent selected from the group consisting of an organic acid solution, inorganic acid solution, acidic buffer, amino acid solution or a mixture thereof.

Preferably, an organic acid is selected from the group consisting of ascorbic acid benzoic acid, a carboxylic acid eg formic acid, acetic acid, propionic acid, butyric acid or valeric acid; a polycarboxylic acid eg citric acid and derivatives and mixtures thereof.

Preferably, an inorganic acid is selected from the group consisting of phosphoric acid, and orthophosphoric acid and derivatives and mixtures thereof.

Buffers are particularly useful in the present invention as the pH can be adjusted in the range of about pH 1 and about pH 6. In one embodiment the acidic buffer is citrophospho buffer or acetate buffer.

In one embodiment, the reagent comprises an amino acid, for example, aspartic acid or glutamic acid.

Preferably, the acid is a week acid. As used herein a weak acid refers to an acid that is not completely dissociated in aqueous solution and accordingly has a relatively low Kₐ value as compared to a strong acid such as hydrochloric acid. Acetic acid, for example, is a weak acid with a dissociation constant of 1.76×10⁻⁵.

In an alternate embodiment, the acid is not a weak acid.

In one embodiment the acid is a dilute acid such that it is prepared at a concentration to be suitable for use according to the present invention. For example, in one embodiment an acid such as orthophosphoric acid is prepared at a concentration of 40 mM.

It is also understood that the pH of an acid can be adjusted as desired. Suitable acidic conditions may be provided by an acid at varying pH's. A person skilled in the relevant art would be familiar with methods to adjust the pH and concentration of an acid to provide suitable conditions for the present invention.

Methods of Recovering a Solubilised Macromolecule.

In one embodiment, the method further comprises precipitating the at least one solubilised macromolecule.

Typically, precipitation is performed using a solvent such as ammonium sulfite, polyethylene glycol (PEG) or by the addition of an organic solvent such as for example, methanol or acetone to the at least one solubilised macromolecule. Preferably, the solvent is added in a ratio of between 4 and 15 times the volume of the sample to be precipitated. In one embodiment the organic solvent is at room temperature, or below room temperature. In one embodiment the sample/solvent mix is allowed to precipitate for approximately 5 minutes to 15 hours. In another embodiment the sample/solvent mix is centrifuged. In a particular embodiment, the sample/solvent mix is centrifuged at about 5000 g for about 10 minutes.

In another embodiment, the method further comprises resuspending be precipitated macromolecule.

In one embodiment the precipitate is resuspended in a reagent having a pH of about pH 1.0 to about pH 6.0. Alternatively the precipitate is resuspended in a solution of neutral pH i.e., about pH 7.0 or at an alkaline pH i.e., pH of greater than about pH 7.0. In one embodiment the solution is resuspended in a solution having pH 10.4.

Analysing Macromolecules

In one embodiment the present invention is useful for solubilising a proteinaceous macromolecule such that it can then be recovered and analysed by proteomic techniques. Preferably, for the purpose of two dimensional electrophoresis a macromolecule that has been recovered according to a method of the invention, is subjected to reduction and alkylation.

Preferably, reducing and alkylating the at least one resuspended macromolecule comprises treating the at least one resuspended macromolecule with one or more agents to reduce and alkylate one or more macromolecules in the sample. Preferably, the one or more reducing or alkylating agents, are selected from the group consisting of dithiothreitol (DTT), Tri-n-butylphosphine (TBP), beta-mercaptoethanol, iodoacetamide, vinyl pyridine, acrylamide, and iodoacetic acid or a mixture thereof.

In one embodiment, the resuspended macromolecule is treated with Tri-n-butylphosphine (TBP). In a preferred embodiment, the sample is treated with Tri-n-butylphosphine (TBP), and then treated with acrylamide.

In a particular embodiment the resuspended macromolecule is treated with 5 mM TBP. In another embodiment, the resuspended macromolecule is treated with 10 mM acrylamide. Preferably, excess acrylamide is quenched with the addition of dithiothreitol (DTT).

It is to be understood that methods of preparing a biological sample including precipitation, resuspension, reduction and alkylation are well known in the art. Various agents are known to the skilled artisan in this regard and are included within the scope of the invention.

Preferably the macromolecule is resolved using proteomic techniques. Many different proteomic techniques are well known to the person skilled in the field of analysing macromolecules Detailed descriptions of proteomics methods can be found for example at the BioBencHelper website which provides a comprehensive collection of internet resources for bio-lab researches.

Preferably, resolving or analysing the at least one macromolecule comprises any of the following proteomic techniques:

two-dimensional electrophoresis, one-dimensional electrophoresis, HPLC or liquid chromatography-mass spectrometry (LC-MS). In particular, mass spectrometry can be used to identify macromolecules.

In a preferred embodiment, the method further comprises digesting the resolved macromolecules; and more preferably, identifying the digested macromolecules by mass-spectrometry.

Preferably, the macromolecules are digested by proteolytic enzymes.

Kits

In a second aspect the invention provides kits for carrying out the methods of the invention.

Accordingly, a variety of kits are provided in suitable packaging. The kits may be used for any one or more of the methods or uses described herein, and accordingly, may contain instructions for any one or more of the following uses: solubilising, recovering, resolving and/or analysing a proteinaceous macromolecule in a biological sample.

Preferably, the kit comprises a solubilisation reagent having a pH of between about pH 1.0 and about pH 6.0 which is capable of under suitable conditions to solubilise a proteinaceous macromolecule in a biological sample such that acid hydrolysis does not substantially occur.

Preferably, the solubilisation reagent comprises a chaotropic agent (e.g. urea or thiourea, or a mixture thereof) and/or a detergent (e.g. ionic detergent, anionic detergent, non-ionic detergent).

In an alternate embodiment, an acidic agent is provided separately to the solubilisation reagent.

In one embodiment, the kit further comprises an agent for disrupting the biological sample, eg., toluene. In another embodiment the kit further comprises an agent for inactivating or removing interfering substances eg., a protease inhibitor. In a further embodiment, the kit includes a reducing and/or alkylating agent.

In one embodiment, the kit comprises a means for resolving at least one macromolecule in the biological sample. In one embodiment, the means for resolving at least one macromolecule comprises an electrophoresis gel.

The kits of the invention comprise one or more containers comprising any one of the reagents, for example, an acidic reagent or solubilisation reagent, as described herein. Each component if there is more than one component can be packaged in separate containers or some components can be combined in one container where cross-reactivity and shelf life permit. The kits of the invention may optionally include a set of instructions, generally written instructions, although electronic storage media containing instructions are also acceptable, relating to the use of components of the methods of the present invention. The instructions included with the kit generally contain information as to the components and their administration to an individual.

In a preferred embodiment the present invention provides an extraction or resuspension reagent having a pH of about pH 1 to about pH 6, wherein the reagent is capable, under suitable conditions, of solubilising a proteinaceous macromolecule without substantial acid hydrolysis of the macromolecule. Preferably, the reagent comprises an aqueous solvent such as an acidic aqueous solvent selected from the group consisting of an organic acid solution, inorganic acid solution, acidic buffer, amino acid solution, or a mixture thereof. Preferably, the reagent further comprises a chaotropic agent or detergent or mixture thereof.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### INDUSTRIAL APPLICABILITY

The method and reagents described herein have significant implications for obtaining many medically, agriculturally and environmentally significant proteins from microorganisms.

The wealth of genome sequences available from microorgaisms and the relative ease of genetic or environmental manipulation of microbial systems make them excellent candidates to produce and recover proteins.

Further improvements in sample preparation for proteomic separation techniques will enable the resolution, identification and study of a greater number of the more insoluble proteins. Microorganisms are excellent models to further our understanding of complex biological systems and the method provided will substantially aid the proteomic study of many biological properties of microorganisms having complex cell walls, often exceedingly more complex than is found in laboratory strains.

### METHODS OF THE INVENTION

Materials and methods

1.1 Materials

Tributylphosphine (TBP) was obtained from Fluka Chemie (Buchs, Switzerland), ProteoPrep™ kits (Prot-Tot), Fungal and Yeast Protease Inhibitor Cocktail # P8215, PMSF, lyophilized Escherichia coli K12 and Saccharomyces cerevisiae were from Sigma, St. Louis, Mo., USA. Cells of Bacillus subtilis SU8 were obtained mid-exponential cultures grown in Luria Broth at 37° C. with good aeration washed with 10 mM Tris, 1 mM EDTA, and stored frozen as wet pellets. Mycelia of Trichoderma harzianum A3091 (IMI 206040) were grown at 28° C. on a shaker at 250 rpm for 54 h. Fungal and Yeast Protease Inhibitor Cocktail (0.05% v/v) was added to the culture and allowed to incubate at room temperature for 20 min. The mycelia were filtered through Whatman 3MM paper (Whatman, Maidstone, England) and washed with 150-200 mL of Milli-Q (18.2 M Ohms) water. Mycelia were collected and either used immediately or stored at −20° C. until use. Membrane centrifugal filters (5 kDa or 100 kDa pore size) were from Millipore, MA, USA.

2.1 Acidic Sample Preparation

Dry or wet E. coli, S. cerevisiae, B. subtilis or T. harzianum cells were solubilised by ultrasonication in 7 M urea, 2 M thiourea, 1% C7Bz0 and either 80 mM citric acid (pH 4), 80 mM ascorbic acid (pH 4), orthophosphoric acid (pH 3) or orthophosphoric acid (pH 2). In some cases acidic sample preparations were done with 1 mM PMSF and 0.1% protease inhibitor cocktail. The sample was maintained below 35° C. during the sonication. The extraction solution was kept on ice and ultrasonicated for 4 burst of 30 seconds with 1 minute cooling between bursts. After centrifugation at 40,000 g for 20 minutes, the supernatant protein was precipitated by the addition of 5 volumes of room temperature acetone. The precipitated proteins were recovered by centrifugation at 5000 g for 10 minutes and resuspended in Sigma ProteoPrep™ (Prot-Tot) extraction solution No. 4, pH 10.4. This solution is 7 M urea, 2 M thiourea, 1% C7Bz0 and 40 mM Tris. The now alkaline protein extract was reduced with 5 mM TBP and alkylated with 10 mM acrylamide monomer in a single 2 hour step. The excess acrylamide was then quenched with the addition of 10 mM DTT, and the sample aliquoted prior to storage at either −20° or −80° C. The final extract contained between 2-3 mg/mL of protein.

2.2 Conventional Alkaline Extraction

Dry or wet E. coli, S. cerevisiae, B. subtilis or Trichoderma harzianum cells were solubilised by ultrasonication in Sigma ProteoPrep™ (Prot-Tot) extraction solution No. 4, pH 10.4. This solution is 7 M urea, 2 M thiourea, 1% C7Bz0 and 40 mM Tris. The ultrasonication procedure was performed as described above. After centrifugation at 40,000 g for 20 minutes, the supernatant protein was precipitated, and reduced, alkylated and stored as described above. The final extract contained 2-3 mg/mL of protein. The precipitation step, whist not technically necessary, was done to ensure both alkaline and acidic extracts were treated in the same way.

2.3 2D Electrophoresis

The protein extracts were loaded by rehydration (250 μL) onto 11 cm pH 3-10 ProteoGel™ IPG strips from Sigma (St. Louis, Mo.), or 11 cm pH 3-10 IPG strips from Amersham Biosciences (Uppsala, Sweden). IEF was conducted using a 5 hour linear ramp to 10 kV and a further 5 hours at 10 kV, using an IsoelectrIQ² IEF device from Proteome Systems (Sydney, Australia). Second dimension gels were GelChip™ long life tricine chemistry gels available from Proteome Systems (Woburn, Mass.). Gels were stained using GelChip Blue, a modified Neuhoff colloidal Coomassie Brilliant Blue G250 stain from Proteome Systems (Woburn, Mass.).

2.4 100 kDa Membrane Filtration

B. subtilis protein extract prepared by alkaline extraction (2.3, above) was buffer exchanged by 5 kDa cut-off membrane centrifugal filtration to remove urea and thiourea from the sample (due to limited compatibility of urea and thiourea with 1D SDS-PAGE). The buffer exchanged sample was filtered by 100 kDa cut-off membrane centrifugal filtration, giving two fractions: the retentate (>100 kDa fraction); and the eluate (>100 kDa fraction). Two dilutions of the eluate and retentate were run on a 1D SDS-agarose-polyacrylamide (SDS-AgPAGE) gel (1% agarose, 0-6% linear gradient acrylamide), and semi-dry electroblotted onto a PVDF membrane. The PVDF blot was stained with alcian blue.

3. Result and Discussion

3.1 E. coli 2D Gels

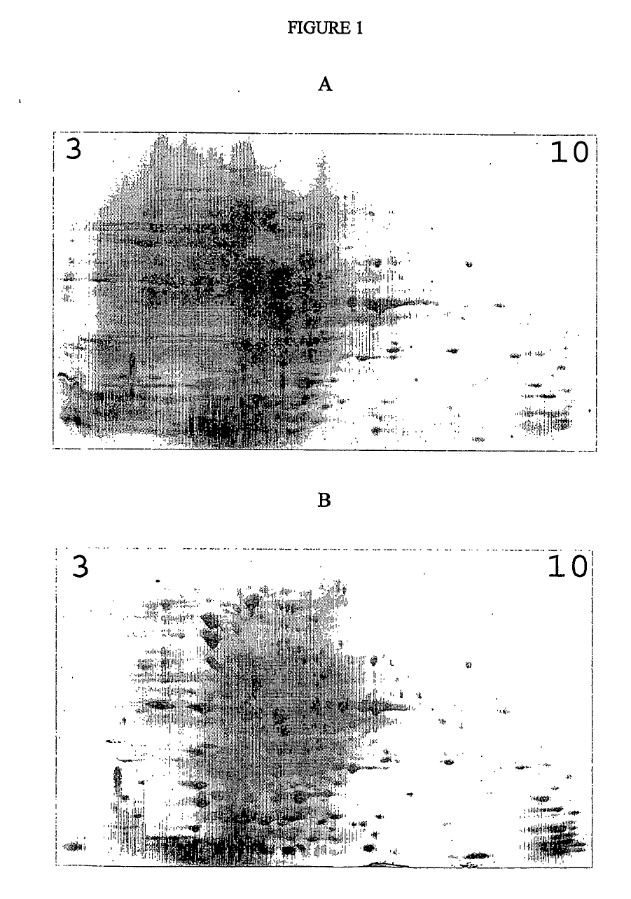

The typical effect of cell wall contamination, as observed on 2D gets of microorganisms, is horizontal streaking in the acidic part of the map. This is clearly shown in FIG. 1a, in which the gram negative E. coli was extracted using conventional alkaline conditions. In contrast FIG. 1b demonstrates a complete removal of streaking when the extraction is performed at acidic pH using citric acid. The removal of the streaking appears to have had no impact on the overall protein distribution on the gel, but has enabled the resolution and detection of many more proteins in the acidic third of the gel.

3.2 B. subtilis 2D Gels

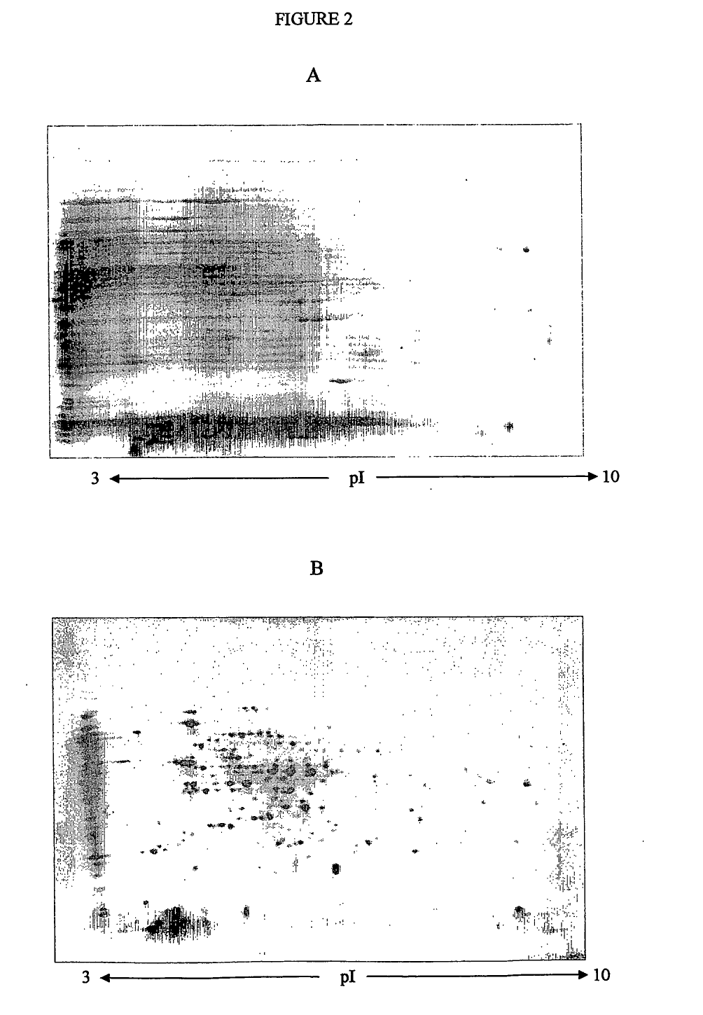

The gram positive B. subtilis provides a major challenge for 2D electrophoresis because of the severe nature of the cell wall associated streaking. The cell wall of gram positive bacteria is typically much larger than in gram negative bacteria, and in B. subtilis often accounts for more than 50% of dry cell weight. The cell wall components that complicate 2D procedures are most likely large, acidic molecules, such as peptidoglycan and teichoic acids. This problem usually necessitates cup loading at the anode to minimize the entry of polysaccharides into the gel. However, cup loading also restricts the amount of protein which can be loaded, and it is preferred to use rehydration loading where possible. FIG. 2a demonstrates the type of result that is consistently obtained when alkaline extracted B. subtilis is rehydration loaded. The majority of the gel is obscured with severe horizontal streaking and only a small number of well resolved protein spots are visible, FIG. 2b is a rehydration loaded gel of B. subtilis extracted under acidic conditions using citric acid. There is a large increase in the number of well resolved spots and horizontal streaking has been eliminated.

3.3 S. cerevisiae 2D Gels

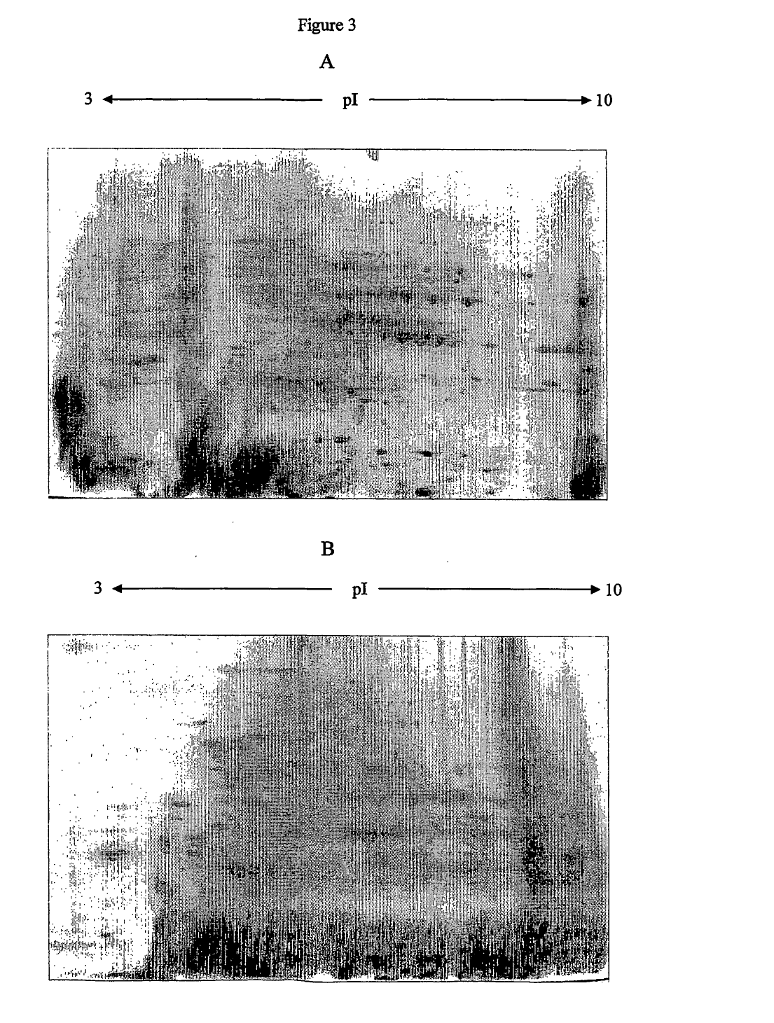

In S. cerevisiae, the cell wall makes up 15 to 30% of the dry weight of the cell and 25 to 50% of the volume based on calculations from electron micrographs. The walls are composed mostly of mannoproteins fibrous β1,3 glucan, chitin and branched β1,6 glucan that links the other components of the wall. On the outer surface of the wall are densely packed mannoproteins, which are extensively O and N glycosylated and limit cell wall permeability to solutes. With such a large proportion of the starting material made up of cell wall it is particularly important to address polysaccharide solubility to limit adverse effects on the gel. As was the case with bacteria, acidic extraction was successful in eliminating streaking associated with cell wall material from S. cerevisiae. The acidic extraction has also significantly increased yield of extracted protein. FIGS. 3a and 3b show S. cerevisiae extracted under conventional alkaline conditions and using 80 mM citric acid respectively. In FIG. 3a the cell wall material has bound detergent causing the large smears observed in the lower left quadrant of the gel and considerable horizontal streaking is also visible in the acidic third of the gel. In FIG. 3b the detergent smears are greatly reduced and the horizontal streaking has been eliminated enabling many more spots to be observed in the acidic portion of the gel, and the yield of alkaline proteins is significantly improved.

3.4 T. harzianum 2D Gels

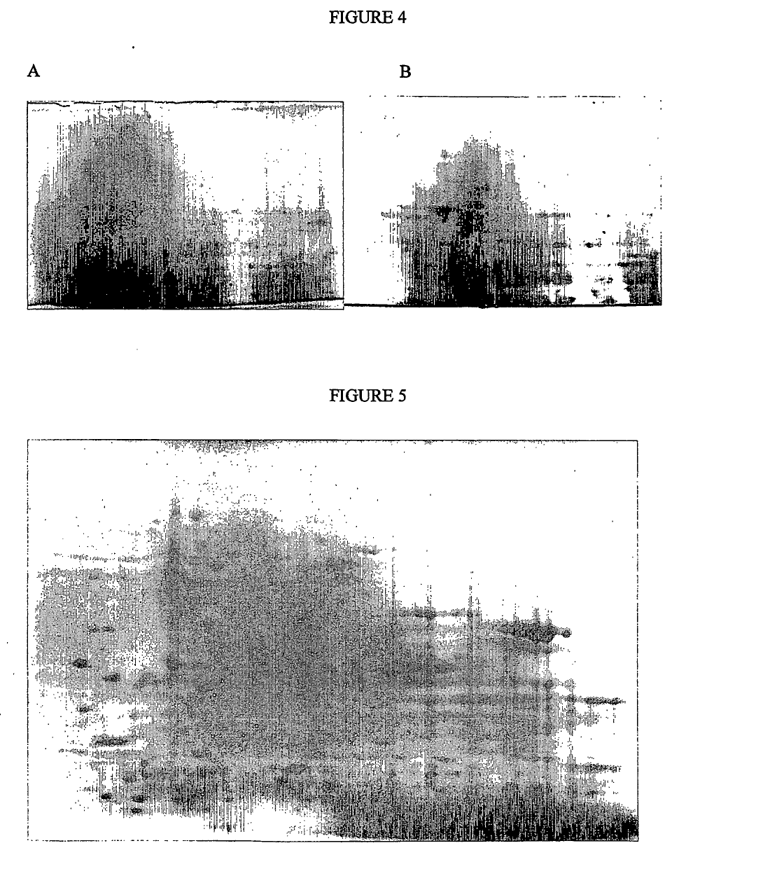

FIGS. 4a and 4b show T. harzianum extracted under conventional alkaline conditions and using 80 mM citric acid respectively. The acidic extraction conditions have successfully eliminated much of the streaking observed with alkaline extraction. FIG. 5 shows the further improved pattern that occurs if acidic extraction of T. harzianum is performed with protease inhibitors. With the other microorganisms studied there was no significant degradation observed when inhibitors were not used during acidic extraction.

3.5 Comparison of Different Acids

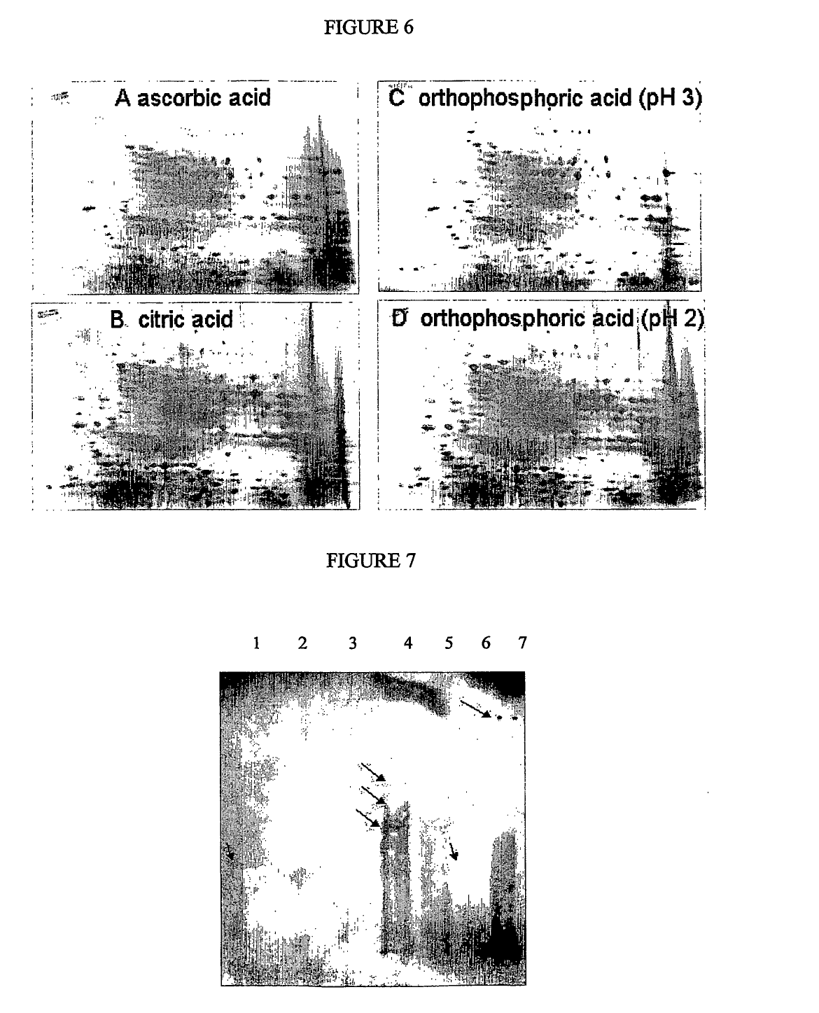

After observing the beneficial effects of acidic extraction, using citric acid, with all four microorganisms tested we went on to investigate ascorbic and orthophosphoric acids to gauge their effectiveness in comparison. FIGS. 6a-6d show a comparison of S. cerevisiae 2D gels, a: 80 mM ascorbic acid, b: 80 mM citric acid, c: orthophosphoric acid pH 3 and d: orthophosphoric acid pH 2. All acids and concentrations were effective at eliminating cell wall associated streaking, however, 80 mM citric acid (pH 4) and orthophosphoric acid at pH 2 produced the highest quantitative yield of protein.

3.6 100 kDa Membrane Filtration

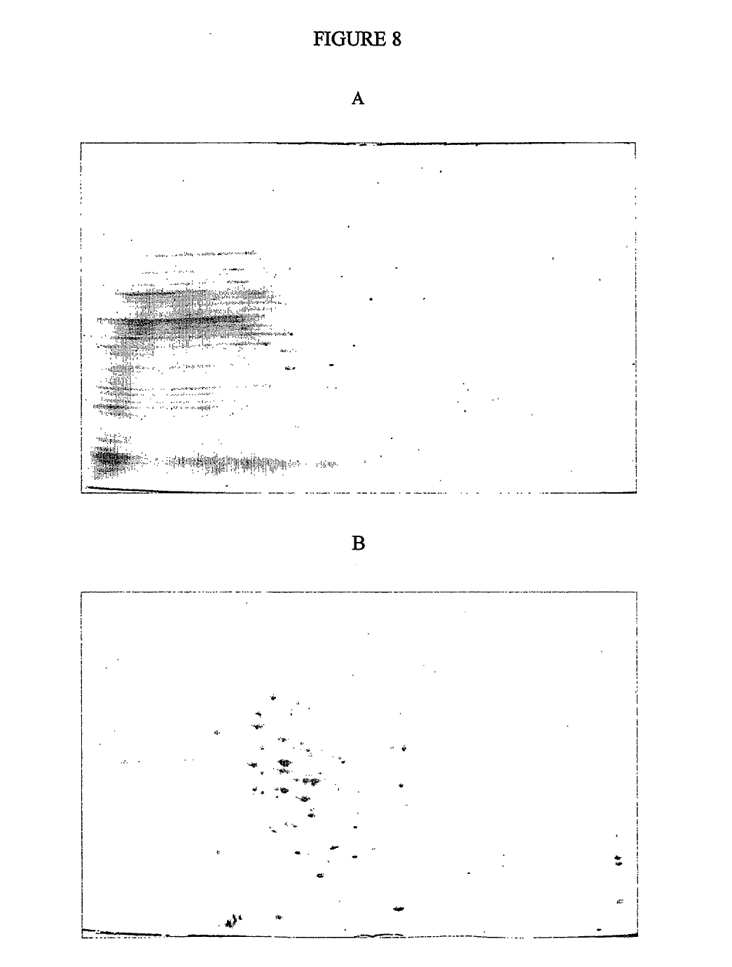

To confirm the identity of the interfering contaminants, a B. subtilis extract was separated into >100 kDa and <100 kDa fractions by membrane filtration. An Alcian Blue stained semi-dry electroblot of a 1D SDS-AgPAGE of these fractions confirms that the >100 kDa fraction contains abundant large, acidic molecules, absent in the <100 kDa fraction (FIG. 7). 2D SDS-PAGE of these demonstrates that the >100 kDa fraction (FIG. 8a) displays a similar pattern to the unfractionated extract (FIG. 2a). The <100 kDa extract (FIG. 8b) has good resolution, confirming that the interfering contaminants are contained in the >100 kDa fraction.

1. A method for enhancing the solubilisation of at least one proteinaceous macromolecule in a biological sample without inducing substantial acid hydrolysis of said proteinaceous macromolecule, said method comprising incubating the biological sample in a solubilisation reagent at a pH between about pH 1.0 and about pH 6.0.

2. The method according to claim 1, wherein the solubilisation reagent has a pH of between about pH 1-0 and about pH 6.0.

3. The method according to claim 1, wherein the biological sample is incubated in a solubilisation reagent at a pH of between about pH 2 to about pH 5.

4. The method according to claim 1, wherein the biological sample is incubated in a solubilisation reagent at a pH of between about pH 3 to about pH 4.

5. The method according to claim 1, wherein the biological sample is incubated in a solubilisation reagent at a pH of between about pH 2 to about pH 3.

6. The method according to claim 1, wherein the proteinaceous macromolecule is solubilised in the presence of an aqueous acidic reagent selected from the group consisting of an organic acid solution, inorganic acid solution, acidic buffer, amino acid solution or a mixture thereof

7. The method according to claim 6, wherein the organic acid is selected from the group consisting of an ascorbic acid, carboxylic acid and polycarboxylic acid, or a derivative or mixture thereof.

8. The method according to claim 7, wherein the carboxylic acid is selected from the group consisting of formic acid, acetic acid, propionic acid, butyric acid, valeric acid, and benzoic acid, or a derivative or mixture thereof.

9. The method according to claim 7, wherein the polycarboxylic acid is selected from the group consisting of oxalic acid and citric acid or a derivative or mixture thereof.

10. The method according to claim 6, wherein the inorganic acid is selected from the group consisting of phosphoric acid, and orthophosphoric acid or a derivative or mixture thereof.

11. The method of claim 6, wherein the acidic buffer comprises acitrophospho buffer.

12. The method of claim 1 wherein the solubilisation reagent comprises a chaotropic agent.

13. The method of claim 1 wherein the solubilisation reagent comprises a detergent.

14. The method according to claim 1 wherein the biological sample is subjected to a physical or chemical means to disrupt the biological sample.

15. The method according to claim 1, further comprising recovering the at least one solubilised proteinaceous macromolecule.

16. The method according to claim 15, wherein the solubilized proteinaceous macromolecule is recovered by performing a process comprising precipitating the at least one solubilised macromolecule.

17. The method according to claim 1, wherein the solubilized proteinaceous macromolecule is recovered by performing a process comprising precipitating and resuspending the protein precipitate.

18. The method according to claim 1 further comprising reducing and alkylating the resuspended protein precipitate.

19. A method of solubilising at least one proteinaceous macromolecule in a biological sample without inducing substantial acid hydrolysis of said proteinaceous macromolecule, said method comprising:
(i) subjecting the biological sample to a physical or chemical means to disrupt said biological sample and incubating the biological sample in the presence of a reagent at a pH between about pH 1.0 and about pH 6.0 to thereby solubilize at least one proteinaceous macromolecule in the biological sample; and
(ii) performing one or more processes selected from the group consisting of:
(a) recovering the solubilized proteinaceous macromolecule by performing a process comprising precipitating one or more proteins in the extract at (i) to thereby precipitate at least the solubilised poteinaceous macromolecule and resuspending the precipitated proteinaceous macromolecule;
(b) reducing and alkylating the solubilized proteinaceous macromolecule at (i) or the resuspended proteinaceous macromolecule at (ii)(a); and
(c) subjecting the solubilized proteinaceous macromolecule at (i) or the resuspended proteinaceous macromolecule at (ii)(a) or the reduced and alkylated proteinaceous macromolecule at (ii)(b) to a resolving means for a time and under conditions sufficient to resolve the proteinaceous macromolecule from other macromolecules present in the biological sample and then identifying the resolved proteinaceous macromolecule.

20. A method of solubilising at least one proteinaceous macromolecule in a biological sample without inducing substantial acid hydrolysis of said proteinaceous macromolecule, said method comprising:
(i) subjecting the biological sample to a physical or chemical means to disrupt said biological sample, thereby producing a proteinaceous extract;
(ii) incubating the proteinaceous extract in the presence of a reagent having a pH between about pH 1.0 and about pH 6.0 to thereby solubilize at least one proteinaceous macromolecule in the extract; and
(iii) performing one or more processes selected from the group consisting of:
(a) recovering the solubilized proteinaceous macromolecule by performing a process comprising precipitating one or more proteins in the extract at (ii) to thereby precipitate at least the solubilised proteinaceous macromolecule and resuspending the precipitated proteinaceous macromolecule;
(b) reducing and alkylating the solubilized proteinaceous macromolecule at (ii) or the resuspended proteinaceous macromolecule at (iii)(a); and
(c) subjecting the solubilized proteinaceous macromolecule at (ii) or the resuspended proteinaceous macromolecule at (iii)(a) or the reduced and alkylated proteinaceous macromolecule at (iii)(b) to a resolving means.

21. The method according to claims 19, wherein the resolving means comprises a proteomic technique selected from the group consisting of: two-dimensional electrophoresis, one-dimensional electrophoresis, HPLC and liquid chromatography-mass spectrometry (LC-MS) or a combination thereof.

22. The method according to claim 19, further comprising the following steps:
iv) digesting the resolved macromolecule, and
v) identifying the digested macromolecule by mass-spectrometry.

23. The method according to claim 22, wherein the macromolecule is digested by at least one proteolytic enzyme.

24. A kit for enhancing solubilisation of a proteinaceous macromolecule a biological sample without inducing substantial acid hydrolysis of said proteinaceous macromolecule, the kit comprising a solubilisation reagent to solubilise at least one macromolecule in a biological sample, wherein the solubilisation reagent has a pH of about pH 1 to about pH 6 and optionally comprising directions to solubilise and/or recover a macromolecule in a biological sample, and/or directions to resolve a macromolecule in a biological sample.

25. A proteinaceous macromolecule solubilised by the method according to claim 1.

26. Use of an acidic reagent having a pH of about pH 1 to about pH 6 in the preparation of an solubilisation reagent solution for use in solubilising a proteinaceous macromolecule from a biological sample, without inducing substantial acid hydrolysis of said proteinaceous macromolecule.
